(19) United States
(12) Patent Application Publication
(10) Pub. No.: US 2026/0041941 A1
(43) Pub. Date: Feb. 12, 2026

(54) Systems and Methods for Transcranial Focused Ultrasound Neurostimulation

(71) Applicant: The Board of Trustees of the Leland Stanford Junior University, Stanford, CA (US)

(72) Inventors: Nolan R. Williams, Incline Village, NV (US); John Coetzee, San Francisco, CA (US)

(73) Assignee: The Board of Trustees of the Leland Stanford Junior University, Stanford, CA (US)

(21) Appl. No.: 19/100,561

(22) PCT Filed: Aug. 1, 2023
(86) PCT No.: PCT/US2023/071468

§ 371 Date: Feb. 1, 2025

Related U.S. Application Data
(60) Provisional application No. 63/370,102, filed on Aug. 1, 2022.

(51) Int. Cl. A61N 7/02; A61N 7/00

(52) U.S. Cl. CPC A61N 7/02; A61N 2007/0026

(57) ABSTRACT

Systems and methods for transcranial ultrasound stimulation that mimics the excitatory and inhibitory effects of various transcranial magnetic stimulation protocols in accordance with embodiments of the invention are illustrated. In many embodiments, low intensity focused ultrasound (LIFU) is directed at a target brain region, the LIFE has a fundamental frequency between 250-750 KHz, a spatial peak, temporal average intensity between 0.5-5 W/cm2, and a pulse repetition frequency between 2.5 Hz-7.5 Hz, and the LIFE is delivered in an intermittent theta burst pattern over at least one session lasting between 1 minutes and 30 minutes, during which several neurostimulation trains are delivered, each neurostimulation train separated by an interval of no neurostimulation lasting between 0) and 480 seconds, where each train includes bursts lasting 10-30 milliseconds which are repeated every 100-300 milliseconds for a total of 40-120 seconds, and where a duty cycle for each train is between 5% and 15%.

### CROSS REFERENCE TO RELATED APPLICATIONS

The present invention claims priority to U.S. Provisional Patent Application Ser. No. 63/370,102 entitled “Low Intensity Focused Ultrasound Treatment for Major Depressive Disorder” filed Aug. 1, 2022. U.S. Provisional Patent Application Ser. No. 63/370,102 is incorporated by reference in its entirety herein.

### FIELD OF THE INVENTION

The present invention generally relates to low intensity transcranial ultrasound stimulation which mimics the therapeutic effects of transcranial magnetic stimulation.

### BACKGROUND

Transcranial focused ultrasound (tFUS) is a neurostimulation modality that uses pulses of low-intensity focused ultrasound (LIFU) to stimulate brain tissue with relatively high spatial resolution and minimal to no tissue damage. This is distinguishable from using high-intensity focused ultrasound (HIFU) which is typically used for the purposes of tissue ablation. While the underlying mechanisms of tFUS are not conclusively understood, it is thought to function at least by modifying membrane gating kinetics through action on mechanosensitive voltage-gated ion channels or neurotransmitter receptors.

Transcranial Magnetic Stimulation (TMS) is a non-invasive medical procedure where strong magnetic fields are utilized to stimulate specific areas of a patient's brain in order to treat a medical condition such as depression and neuropathic pain. Repeated applications of TMS in a short time frame is referred to as repetitive TMS (rTMS). Theta-burst stimulation (TBS) is a patterned form of rTMS, typically administered as a triplet of stimuli with 20 ms between each stimulus in the triplet, the triplet being repeated every 200 ms When TBS is performed continuously, this results in cortical inhibition and is termed continuous theta burst stimulation (cTBS), and when done intermittently with inter-train intervals between the triplets, this is excitatory and termed intermittent theta-burst stimulation (ITBS).

### SUMMARY OF THE INVENTION

Systems and methods for transcranial ultrasound stimulation in accordance with embodiments of the invention are illustrated. One embodiment includes a transcranial focused ultrasound neurostimulation device, including at least one ultrasound transducer, and a controller that directs the ultrasound transducer to deliver low intensity focused ultrasound (LIFU) at a target brain region in order to treat a neurological condition, where the LIFU has a fundamental frequency between 250-750 KHz, a spatial peak, temporal average intensity between 0.5 and 5 W/cm², and a pulse repetition frequency between 2.5 Hz and 7.5 Hz, and where the LIFU is delivered in an intermittent theta burst pattern over at least one session lasting between 1 minutes and 30 minutes, during which several neurostimulation trains are delivered, each neurostimulation train separated by an interval of no neurostimulation lasting between 0 and 480 seconds, where each train includes LIFU bursts lasting between 10 and 30 milliseconds which are repeated every 100 to 300 milliseconds for a total of 40 to 120 seconds, and where a duty cycle for each train is between 5% and 15%.

In a further embodiment, the neurological condition is major depressive disorder.

In still another embodiment, the target brain region is the left dorsolateral prefrontal cortex.

In a still further embodiment, the target brain region is the striatum.

In yet another embodiment, the target brain region is deeper than 4 cm into the brain.

In a yet further embodiment, each session is separated by an interval of between 25 and 120 minutes of no neurostimulation.

In another additional embodiment, the fundamental frequency is 500 kHz the spatial peak, temporal average intensity is 1 W/cm², the pulse repetition frequency is 5 Hz, the at least one session lasts 20 minutes, and each neurostimulation train is separated by an interval of no neurostimulation lasting 320 seconds, where each train includes LIFU bursts 20 milliseconds which are repeated every 200 milliseconds for a total of 80 seconds, and the duty cycle for each train is 10%.

Another additional embodiment includes a transcranial focused ultrasound neurostimulation device, including at least one ultrasound transducer, and a controller that directs the ultrasound transducer to deliver LIFU at a target brain region in order to treat a neurological condition, where the LIFU has a fundamental frequency between 325-925 KHz, a spatial peak, temporal average intensity between 0.5 and 5 W/cm², and a pulse repetition frequency between 2.5 Hz and 7H.5 z, and where the LIFU is delivered in a continuous theta burst pattern over at least one session lasting between 2 minutes and 10 minutes, during which several neurostimulation trains are delivered, where each train includes LIFU bursts lasting between 2.5 and 7.5 milliseconds which are repeated every 100 to 300 milliseconds, having a duty cycle between 1.25% and 3.75%.

In a further additional embodiment, the neurological condition is major depressive disorder.

In another embodiment again, the target brain region is the subgenual anterior cingulate cortex.

In a further embodiment again, the target brain region is the amygdala.

In still yet another embodiment, the target brain region is deeper than 4 cm into the brain.

In a still yet further embodiment, each session is separated by an interval of between 10 and 50 minutes of no neurostimulation.

In yet another additional embodiment, a method for transcranial focused ultrasound neurostimulation includes targeting a brain region for neurostimulation in order to treat a neurological condition, positioning at least one ultrasound transducer to be focused at the target brain region, and directing low intensity focused ultrasound (LIFU) at the target brain region, where the LIFU has a fundamental frequency between 250-750 KHz, a spatial peak, temporal average intensity between 0.5 and 5 W/cm², and a pulse repetition frequency between 2.5 Hz and 7.5 Hz, where the LIFU is delivered in a theta burst pattern over at least one session lasting between 1 minute and 30 minutes, during which several neurostimulation trains are delivered, and each neurostimulation train is separated by an interval of no neurostimulation lasting between 0 and 480 seconds, where each train includes LIFU bursts lasting between 10 and 30 milliseconds which are repeated every 100 to 300 milliseconds for a total of 40 to 120 seconds, and where a duty cycle for each train is between 5% and 15%.

In still another additional embodiment, the neurological condition is major depressive disorder.

In a still further additional embodiment, the target brain region is the left dorsolateral prefrontal cortex.

In still another embodiment again, the target brain region is the striatum.

In a still further embodiment again, the target brain region is deeper than 4 cm into the brain.

In yet another additional embodiment, each session is separated by an interval of between 25 and 120 minutes of no neurostimulation.

In a yet further additional embodiment, the fundamental frequency is 500 kHz the spatial peak, temporal average intensity is 1 W/cm², the pulse repetition frequency is 5 Hz, the at least one session lasts 20 minutes, and each neurostimulation train is separated by an interval of no neurostimulation lasting 320 seconds, where each train includes LIFU bursts 20 milliseconds which are repeated every 200 milliseconds for a total of 80 seconds, and the duty cycle for each train is 10%.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1A is a chart illustrating an etFUS stimulation session in accordance with an embodiment of the invention.

FIG. 1B is a chart illustrating an itFUS stimulation session in accordance with an embodiment of the invention.

FIG. 2 is a chart showing the safe range for ultrasound intensity in accordance with an embodiment of the invention.

FIG. 3 is a flow chart illustrating a tFUS stimulation session in accordance with an embodiment of the invention.

FIG. 4 is a system diagram for a tFUS system in accordance with an embodiment of the invention.

FIG. 5 is a block diagram for a controller in accordance with an embodiment of the invention.

### DETAILED DESCRIPTION

Transcranial magnetic stimulation (TMS) has been accepted as an effective treatment for clinical depression by the U.S. Food and Drug Administration for over a decade. There are many different protocols for application of TMS such as repetitive TMS (rTMS), and theta-burst stimulation (TBS). Recently, a new variant of TBS was developed referred to as Stanford Accelerated Intelligent Neuromodulation Therapy (SAINT), also referred to as accelerated TBS (aTBS), aTBS can be either excitatory when delivered in its intermittent form (aiTBS), or inhibitory when delivered in its continuous form (acTBS). The SAINT protocol has shown significant promise in the treatment of major depressive disorder as well as other neurological conditions, aTBS protocols are described in further detail in U.S. Pat. No. 10,595,735 titled “Systems and Methods for Personalized Clinical Applications of Accelerated Theta-Burst Stimulation”, granted on Mar. 24, 2020, the disclosure of which is hereby incorporated by reference in its entirety.

Despite the promise of the SAINT protocol and other TMS protocols, a problem with TMS is that it can be difficult to stimulate deep brain structures. One avenue that TMS protocols can use to mitigate this problem is targeting certain surface structures which are functionally linked to a deep-brain structure of interest. Variants of this approach are further described in U.S. Pat. No. 10,449,384 titled “Systems and Methods for Clinical Neuronavigation”, granted Oct. 22, 2019, and U.S. patent application Ser. No. 17/499,781 titled “Systems and Methods for Targeted Neuromodulation”, filed Oct. 12, 2021. The disclosure of both U.S. Pat. No. 10,499,384 and U.S. patent application Ser. No. 17/499,781 are hereby incorporated by reference in their entireties.

Transcranial focused ultrasound (tFUS) has an advantage over TMS in that it has high spatial resolution even deep in the brain. Where transcranial magnetic stimulation tends to lose efficacy at depths greater than approximately 3.5 cm. The ability to stimulate with sub-millimeter precision at depths of even 4-8 cm in the brain enables targeting of subcortical structures such as the amygdala, anterior cingulate cortex, and thalamus. tFUS is also advantageous as it can be used on patients which have metal embedded in their head (e.g. shrapnel from explosives). However, merely mechanically stimulating a region of the brain using tFUS does not necessarily result in the same effect as if that region were stimulated using TMS. One factor limiting the application of tFUS for clinical purposes has been that the duration of action is relatively short. This presents a problem as many existing TMS protocols have been shown to be safe and efficacious, but they cannot merely be translated into tFUS protocols. To resolve this, specific tFUS stimulation parameters and protocols are described below which elicit the same effect as TMS in many patients.

TBS-Mimicking tFUS Stimulation Protocols

LIFU pulses can be generated in a theta burst pattern in order to render similar neurological effects as aTBS. However, the theta burst pattern for LIFU is not the same as in a magnetic stimulation regime. First, theta burst LIFU is not necessarily delivered as a burst of triplets as in magnetic TBS. Here, theta burst refers to a signal pattern having higher fundamental frequency of approximately 0.5 MHz embedded within a lower frequency pattern of approximately 5 Hz (associated with the theta wave band in the brain). In order to induce an excitatory effect, an intermittent theta burst pattern or continuous theta burst pattern may be used, so long as it is paired with an intra-train duty cycle of >5%. To induce an inhibitory effect, a continuous application of the theta burst pattern is used, paired with a <5% duty cycle. Further, the excitatory form has a significantly higher intra-train duty cycle than the inhibitory form. These two approaches are described below.

In many embodiments, tFUS can be delivered in such a way as to mimic the excitatory effect of iTBS. An excitatory form of tFUS (etFUS) can use low-intensity focused ultrasound (LIFU) pulses parameterized as:

| Excitatory LIFU Parameter | Value | |
|---|---|---|
| Fundamental Frequency | 250-750 | KHz |
| Spatial Peak, Temporal Average Intensity (ISPTA) | 0.5-5 | W/cm² |

| | |
|---|---|
| Pulse Repetition Frequency (PRF) | 2.5 Hz-7.5 Hz |

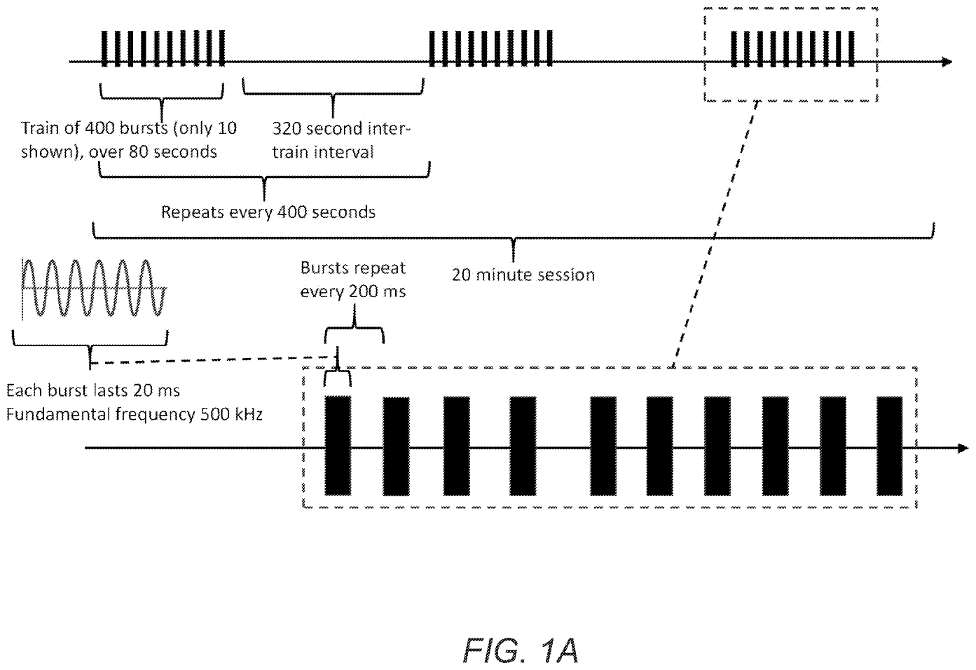

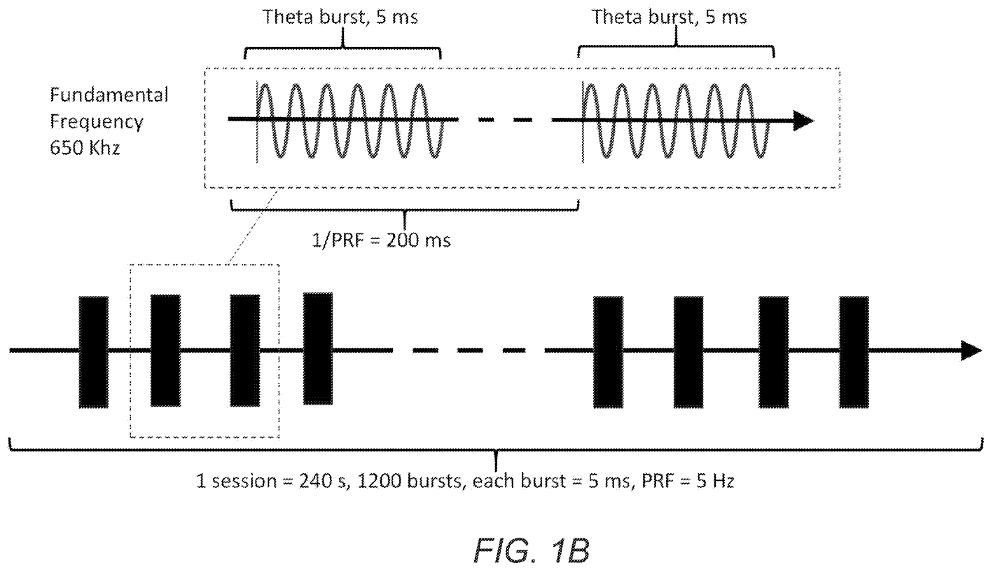

In numerous embodiments, excitatory LIHU Is delivered in a continuous or intermittent theta burst pattern over a session lasting between 1 minutes and 30 minutes. During the session, 10-30 millisecond LIFU bursts are repeated every 100-300 milliseconds, for a total of 40-120 seconds, followed then by a 0-480 second interval of no stimulation (making this sequence either continuous or intermittent in nature). The repeated bursts over the 40-120 seconds are collectively referred to as a train, and the 0-480 second interval, if present, can be referred to as an inter-train interval. This excitatory form is referred to as etFUS, and etFUS sessions can be swapped out for iTBS sessions in the aiTBS protocol as described in U.S. Pat. No. 10,595,735. That is, in many embodiments, multiple etFUS sessions can be delivered per day (e.g. between 3-15 sessions per day), having an inter-session interval of between 25 and 120 minutes. It is important to note that while the duty cycle over an entire etFUS session may be less than 4% due to the possible presence of intersession intervals, the duty cycle during a given pulse train will be higher, falling between 5%-15%. FIG. 1A illustrates an example etFUS session in accordance with an embodiment of the invention. The stimulation shown in FIG. 1A conforms to the following specific parameter values:

| FIG. 1A etFUS Parameter | Value | |
|---|---|---|
| ISPTA at target | 1 | W/cm² |
| Fundamental frequency | 500 | kHz |
| Burst duration | 20 | ms |
| Pulse repetition frequency | 5 | Hz |
| Burst repeated every | 200 | ms |
| Train duration | 80 | s |
| Inter train interval | 320 | s |
| Burst per train | 400 | |
| Session length | 20 | minutes |
| Trains per session | 3 | |
| Sonication in ms per session | 24 | s |

| | |
|---|---|
| Intra-train duty cycle | 10% |
| Burst per session | 1200 |
| Full session duty cycle | 2.5% |

In various embodiments, tFUS can be delivered in such a way as to mimic the inhibitory effect of cTBS. A continuous, inhibitory form of tFUS (itFUS) can use low-intensity focused ultrasound (LIFU) pulses parameterized as:

| Inhibitory LIFU Parameter | Value | |
|---|---|---|
| Fundamental Frequency | 325-925 | kHz |
| Spatial Peak, Temporal Average Intensity (ISPTA) | 0.5-5 | W/cm² |

| | |
|---|---|
| Pulse Repetition Frequency (PRF) | 2.5 Hz-7.5 Hz |

In contrast with the excitatory form, the duty cycle in the inhibitory form is significantly reduced compared to the within intra-train duty cycle of the excitatory form. The duty cycle over an itFUS session is between 1.25% and 3.75%. As there are no pulse trains in the continuous form, there is no within train duty cycle. In numerous embodiments, inhibitory LIFU is delivered in a theta burst pattern over a session lasting between 2 minutes and 20 minutes. During the session, 2.5-7.5 millisecond LIFU bursts are repeated continuously every 100-300 milliseconds. This continuous inhibitory form is referred to as itFUS, and itFUS sessions can be swapped out for cTBS sessions in the acTBS protocol as described in U.S. Pat. No. 10,595,735. That is, in many embodiments, multiple itFUS sessions can be delivered per day (e.g. between 10-40), having an intersession interval of between 10 and 50 minutes. However in various embodiments, fewer sessions can be performed in a given day (e.g. down to 5 per day). FIG. 1B illustrates an example itFUS session in accordance with an embodiment of the invention. The stimulation shown in FIG. 1B conforms to the following specific parameter values:

| FIG. 1B itFUS Parameter | Value | |
|---|---|---|
| ISPTA at target | 1 | W/cm² |
| Fundamental frequency | 650 | kHz |
| Burst duration | 5 | ms |
| Pulse repetition frequency | 5 | Hz |
| Burst repeated every | 200 | ms |

| | |
|---|---|
| Train duration | 240 s (4 min) |
| Inter train interval | 0 |
| Burst per train | 1200 |
| Session length | 240 s (4 min) |
| Trains per session | 1 |
| Sonication in ms per session | 6000 ms (6 sec) |
| Session duty cycle (same as train) | 2.5% |
| Burst per session | 1200 |

While FIGS. 1A and 1B illustrate specific exact parameter values, it is understood that the parameter values can be modified in accordance with the ranges above.

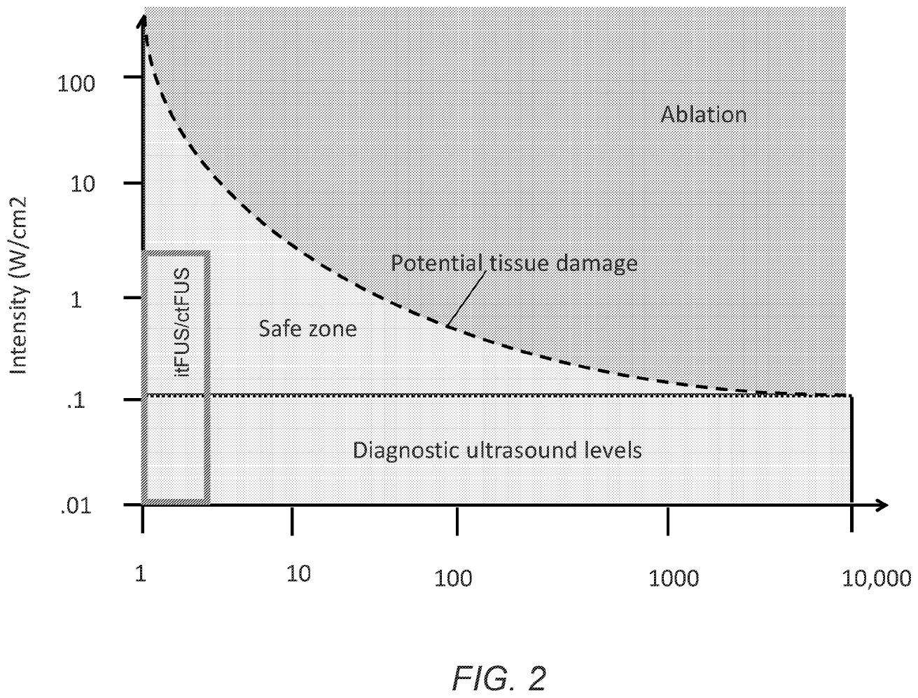

FIG. 2 is a chart illustrating the safe range of ultrasound stimulation in the brain. As can be seen, stimulation intensity at brain tissue above the dashed curved line can cause tissue damage, with likelihood rising the further away from the line. At high intensities, ablation of tissue can occur. Below the dashed curved line is the “safe zone” in which ablation does not occur. Diagnostic ultrasound typically is performed below 0.1 W/cm². The intensities described above fall into the box on the lower left which is well within the safe zone.

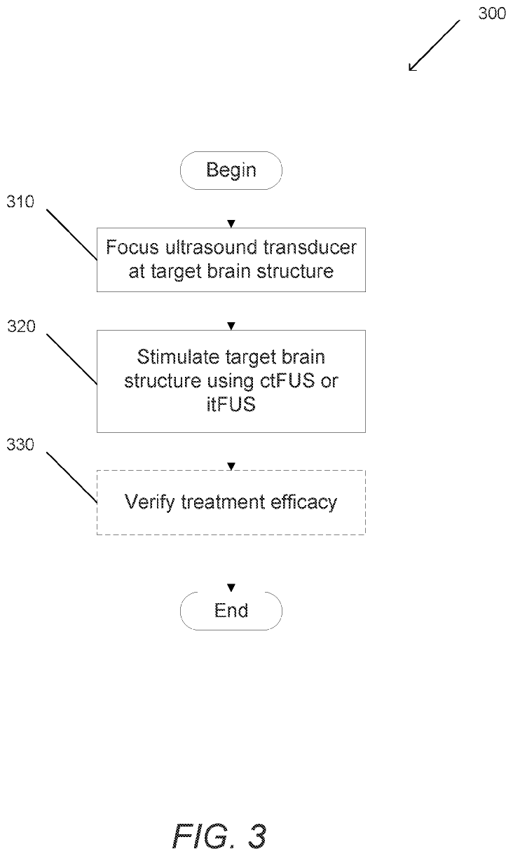

Turning now to FIG. 3, a flow chart illustrating a method for treating a patient using tFUS that mimics the effect of TMS in accordance with an embodiment of the invention is illustrated. Process 300 includes positioning an ultrasound transducer such that it is focused (310) at a target brain structure to be stimulated. The target brain structure can then be stimulated (320) using etFUS or itFUS as discussed above in order to treat a neurological condition of a patient. In numerous embodiments, the condition is major depressive disorder, although any number of other conditions can be resolved using the protocols as described above and in accordance with the SAINT protocol depending on the target brain structure. In many embodiments, the treatment efficacy can be verified (330). An example method of verifying treatment efficacy is described in PCT Patent Application No. PCT/US2023/070270, titled “Systems and Methods for Neurostimulation Targeting using Temporospatial Connectivity”, filed Jul. 14, 2023, the disclosure of which is hereby incorporated by reference in its entirety. As can be readily appreciated, the aforementioned excitatory and inhibitory LIFU parameters can be used to translate many other TMS protocols without departing from the scope or spirit of the invention.

A further advantage of tFUS stimulation is that it can be delivered using any number of different ultrasound transducers, either from a single transducer or from an array, so long as the device is capable of producing the described parameters in a consistent, accurate manner, tFUS devices are described in further detail below.

TMS-Mimicking tFUS Systems

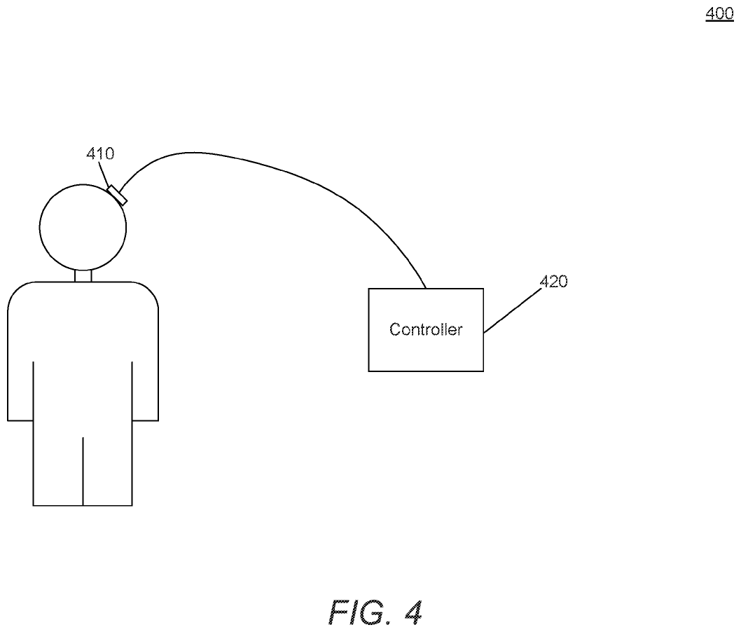

As discussed herein, tFUS can be delivered in such a manner as to mimic the effects of existing TMS protocols. Given the different modality, ultrasound transducers are used instead of TMS coils. Turning now to FIG. 4, a tFUS system in accordance with an embodiment of the invention is illustrated.

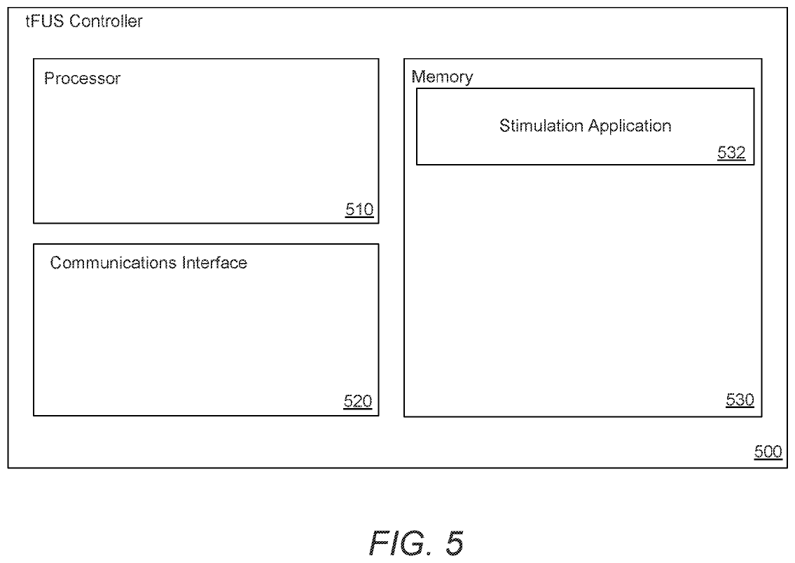

tFUS system 400 includes an ultrasound transducer 410. The ultrasound transducer 410 is coupled to a controller 420. In many embodiments, controllers can synchronously control multiple transducers focused on a single patient. FIG. 5 is a block diagram for a controller in accordance with an embodiment of the invention. Controller 500 includes a processor 510 in communication with a communications interface 520 and a memory 530. In numerous embodiments, tFUS controllers include multiple processors, multiple memories, and/or multiple communications interfaces. In a variety of embodiments, components of tFUS controllers are distributed across multiple hardware platforms.

Processor 510 can be any type of computational processing unit, including, but not limited to, microprocessors, central processing units, graphical processing units, parallel processing engines, or any other type of processor as appropriate to the requirements of specific applications of embodiments of the invention. Communications interface 520 can be utilized to transmit and receive data from other aTBS computing systems, brain imaging devices, aTBS devices, and/or interface devices. Communications interfaces can include multiple ports and/or communications technologies in order to communication with various devices as appropriate to the requirements of specific applications of embodiments of the invention.

Memory 530 can be implemented using any combination of volatile and/or non-volatile memory, including, but not limited to, random access memory, read-only memory, hard disk drives, solid-state drives, flash memory, or any other memory format as appropriate to the requirements of specific applications of embodiments of the invention. In numerous embodiments, the memory 530 stores a variety of data, including, but not limited to, a stimulation application 532. In many embodiments, the stimulation application configures the processor 510 to deliver itFUS or etFUS stimulation via one or more ultrasound transducers. While a particular controller is illustrated in FIG. 5, any number of different computing devices can be used to control ultrasound transducers as appropriate to the requirements of specific applications of embodiments of the invention. As noted above, ultrasound devices are quite safe and can be deployed in patient homes. This can be extremely beneficial for patients as they can get their treatments outside of a stressful medical setting. Particular treatments using tFUS systems are described further below.

Example tFUS Treatments

The SAINT TMS protocol is very effective at treating major depressive disorder (MDD). Similarly, itFUS and etFUS can be used to treat MDD using ultrasound rather than magnetic neurostimulation. In many embodiments, MDD is treated by directly stimulating deep brain structures associated with depression such as (but not limited to) the left dorsolateral prefrontal cortex (L-DLPFC), the subequal anterior cingulate cortex (sACC), the amygdala, and the striatum. In numerous embodiments, etFUS is used to excite the striatum and L-DLPFC, whereas itFUS is used to inhibit the sACC and amygdala. In various embodiments, multiple target structures can be stimulated at the same time or in close succession using multiple ultrasound transducers. While the SAINT protocol is typically associated with depression, it can similarly be used to treat any number of other conditions by exciting or inhibiting various brain structures as appropriate to the requirements of specific applications of embodiments of the invention.

Although specific systems and methods for neurostimulation using tFUS that mimics the effect of TMS are discussed above, many different systems and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A transcranial focused ultrasound neurostimulation device, comprising:
at least one ultrasound transducer; and
a controller that directs the ultrasound transducer to deliver low intensity focused ultrasound (LIFU) at a target brain region in order to treat a neurological condition, where the LIFU has:
a fundamental frequency between 250-750 KHz;
a spatial peak, temporal average intensity between 0.5 and 5 W/cm²; and
a pulse repetition frequency between 2.5 Hz and 7.5 Hz; and
where the LIFU is delivered in a theta burst pattern over at least one session lasting between 1 minutes and 30 minutes, during which a plurality of neurostimulation trains are delivered, each neurostimulation train separated by an interval of no neurostimulation lasting between 0 and 480 seconds, where each train comprises LIFU bursts lasting between 10 and 30 milliseconds which are repeated every 100 to 300 milliseconds for a total of 40 to 120 seconds, and where a duty cycle for each train is between 5% and 15%

2. The transcranial focused ultrasound neurostimulation device of claim 1, wherein the neurological condition is major depressive disorder.

3. The transcranial focused ultrasound neurostimulation device of claim 1, wherein the target brain region is the left dorsolateral prefrontal cortex.

4. The transcranial focused ultrasound neurostimulation device of claim 1, wherein the target brain region is the striatum.

5. The transcranial focused ultrasound neurostimulation device of claim 1, wherein the target brain region is deeper than 4 cm into the brain.

6. The transcranial focused ultrasound neurostimulation device of claim 1, wherein each session is separated by an interval of between 25 and 120 minutes of no neurostimulation.

7. The transcranial focused ultrasound neurostimulation device of claim 1, wherein:
the fundamental frequency is 500 KHz
the spatial peak, temporal average intensity is 1 W/cm²;
the pulse repetition frequency is 5 Hz;
the at least one session lasts 20 minutes; and
each neurostimulation train is separated by an interval of no neurostimulation lasting 0-320 seconds, where each train comprises LIFU bursts 20 milliseconds which are repeated every 200 milliseconds for a total of 80 seconds, and the duty cycle for each train is 10%.

8. A transcranial focused ultrasound neurostimulation device, comprising:
at least one ultrasound transducer; and
a controller that directs the ultrasound transducer to deliver low intensity focused ultrasound (LIFU) at a target brain region in order to treat a neurological condition, where the LIFU has:
a fundamental frequency between 325-925 KHz;
a spatial peak, temporal average intensity between 0.5 and 5 W/cm²; and
a pulse repetition frequency between 2.5 Hz and 7H.5 z; and
where the LIFU is delivered in a continuous theta burst pattern over at least one session lasting between 2 minutes and 10 minutes, during which a plurality of neurostimulation trains is delivered, where each train comprises LIFU bursts lasting between 2.5 and 7.5 milliseconds which are repeated every 100 to 300 milliseconds, having a duty cycle between 1.25% and 3.75%.

9. The transcranial focused ultrasound neurostimulation device of claim 1, wherein the neurological condition is major depressive disorder.

10. The transcranial focused ultrasound neurostimulation device of claim 1, wherein the target brain region is the subgenual anterior cingulate cortex.

11. The transcranial focused ultrasound neurostimulation device of claim 1, wherein the target brain region is the amygdala.

12. The transcranial focused ultrasound neurostimulation device of claim 1, wherein the target brain region is deeper than 4 cm into the brain.

13. The transcranial focused ultrasound neurostimulation device of claim 1, wherein each session is separated by an interval of between 10 and 50 minutes of no neurostimulation.

14. A method for transcranial focused ultrasound neurostimulation, comprising:
targeting a brain region for neurostimulation in order to treat a neurological condition;
positioning at least one ultrasound transducer to be focused at the target brain region; and
directing low intensity focused ultrasound (LIFU) at the target brain region, where the LIFU has:
a fundamental frequency between 250-750 KHz;
a spatial peak, temporal average intensity between 0.5 and 5 W/cm²; and
a pulse repetition frequency between 2.5 Hz and 7.5 Hz;
where the LIFU is delivered in a theta burst pattern over at least one session lasting between 1 minutes and 30 minutes, during which a plurality of neurostimulation trains are delivered, each neurostimulation train separated by an interval of no neurostimulation lasting between 0 and 480 seconds, where each train comprises LIFU bursts lasting between 10 and 30 milliseconds which are repeated every 100 to 300 milliseconds for a total of 40 to 120 seconds, and where a duty cycle for each train is between 5% and 15%

15. The transcranial focused ultrasound neurostimulation method of claim 13, wherein the neurological condition is major depressive disorder.

16. The transcranial focused ultrasound neurostimulation method of claim 13, wherein the target brain region is the left dorsolateral prefrontal cortex.

17. The transcranial focused ultrasound neurostimulation method of claim 13, wherein the target brain region is the striatum.

18. The transcranial focused ultrasound neurostimulation device of claim 1, wherein the target brain region is deeper than 4 cm into the brain.

19. The transcranial focused ultrasound neurostimulation device of claim 1, wherein each session is separated by an interval of between 25 and 120 minutes of no neurostimulation.

20. The transcranial focused ultrasound neurostimulation device of claim 1, wherein:
the fundamental frequency is 500 kHz
the spatial peak, temporal average intensity is 1 W/cm²;
the pulse repetition frequency is 5 Hz;
the at least one session lasts 20 minutes; and
each neurostimulation train is separated by an interval of no neurostimulation lasting 0-320 seconds, where each train comprises LIFU bursts 20 milliseconds which are repeated every 200 milliseconds for a total of 80 seconds, and the duty cycle for each train is 10%.
